(19) United States
(12) Patent Application Publication
(10) Pub. No.: US 2019/0105470 A1
(43) Pub. Date: Apr. 11, 2019

(54) SHEATHS, METHODS OF USE, AND KITS INCLUDING THE SAME

(71) Applicant: Baylor College of Medicine, Houston, TX (US)

(72) Inventors: Naveen Yadav, Houston, TX (US); Arun Rai, Houston, TX (US); Christopher Graziano, Houston, TX (US); Samit Soni, Houston, TX (US); Richard Link, Bellaire, TX (US)

(73) Assignee: Baylor College of Medicine, Houston, TX (US)

(21) Appl. No.: 16/088,092

(22) PCT Filed: Apr. 6, 2017
(86) PCT No.: PCT/US2017/026283

§ 371 Date: Sep. 25, 2018

Related U.S. Application Data
(60) Provisional application No. 62/322,449, filed on Apr. 14, 2016.

(51) Int. Cl. A61M 25/06; A61M 25/10; A61M 25/09

(52) U.S. Cl. CPC A61M 25/0662; A61M 25/10; A61M 2210/1082; A61M 25/09; A61M 25/10186

(57) ABSTRACT

One aspect of the invention provides a sheath including: a cylinder defining a central passage; a balloon located along an external surface of a distal end of the cylinder, the balloon lying substantially flush with or recessed from the external surface when deflated, but capable of projecting beyond the external surface when inflated; and an inflation port located at a proximal end of the cylinder, the inflation port in fluid communication with the balloon. Another aspect of the invention provides a kit including: a sheath as described herein and instructions for use.

### CROSS-REFERENCE TO RELATED APPLICATION

This application claims priority to U.S. Provisional Patent Application Ser. No. 62/322,449, filed Apr. 14, 2016. The entire content of the application is hereby incorporated by reference herein.

### BACKGROUND OF THE INVENTION

The percutaneous nephrolithotomy (PCNL) procedure is a surgery performed by urologists for the treatment of large kidney stones (usually >2 cm). The procedure has a success rate exceeding 90% in rendering patients stone free, making it an important technique in the modern-day urologists' arsenal.

### SUMMARY OF THE INVENTION

One aspect of the invention provides a sheath including: a cylinder defining a central passage; a balloon located along an external surface of a distal end of the cylinder, the balloon lying substantially flush with or recessed from the external surface when deflated, but capable of projecting beyond the external surface when inflated; and an inflation port located at a proximal end of the cylinder, the inflation port in fluid communication with the balloon.

This aspect of the invention can have a variety of embodiments. The sheath can further include a locking member slidably positioned along the external surface of the cylinder and adapted and configured to press against a subject's skin. The sheath can further include a one-way valve in communication with the inflation port, the one-way valve adapted and configured to retain a fluid and maintain the balloon in an inflated state.

The distal end can be beveled with respect to a central axis of the cylinder. The distal end can be beveled with respect to the external surface of the cylinder.

The cylinder can have a cylindrical cross-section.

The central passage can have a diameter of about 26 French or greater. The central passage can have a diameter of about 34 French or greater.

The balloon can be fabricated from a material selected from the group consisting of: polyurethane, polyvinylchloride, latex, guayule latex, and silicone rubber.

Another aspect of the invention provides a method of performing a percutaneous procedure. The method includes: inserting a guidewire into an object of interest; dilating an opening defined by the guidewire; inserting a sheath as described herein into the opening; advancing the sheath so that the balloon enters the object of interest; and introducing a fluid into the inflation port to inflate the balloon, thereby retaining the sheath within the distal end of the sheath within the object of interest.

Another aspect of the invention provides a method of performing a percutaneous nephrolithotomy (PCNL) procedure. The method includes: inserting a guidewire into a renal collecting system of a subject's kidney; dilating an opening defined by the guidewire; inserting a sheath as described herein into the opening; advancing the sheath so that the balloon enters the renal collecting system; and introducing a fluid into the inflation port to inflate the balloon, thereby retaining the sheath within the distal end of the sheath within the renal collecting system.

Another aspect of the invention provides a kit including: a sheath as described herein and instructions for use.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A and 1B provide photographs of a sheath according to an embodiment of the invention.

FIGS. 2A-2E provide line drawings of a sheath according to an embodiment of the invention.

FIG. 3 depicts a method of a method of performing a percutaneous nephrolithotomy (PCNL) procedure according to an embodiment of the invention.

FIG. 4 depicts the inflation of the balloon of a sheath within a model of a kidney according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

The term “cylinder” not only includes three-dimensional shapes having a circular cross-section, but also any surface consisting of each of the straight lines that are parallel to a given straight line and pass through a given curve.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Briefly, the percutaneous nephrolithotomy (PCNL) procedure involves traversing the flank tissue in order to gain percutaneous access to the renal collecting system. Once access is gained, the tract into the kidney is serially dilated to approximately 26-30 French to allow for passage of a rigid nephroscope and various lithotriptors. A standard access sheath is generally inserted over a wire into the access site and used to provide a continuous passage into the collecting system for introduction and exchange of scopes and lithotripters during the case.

Surgical complications occurring during or after PCNL have been reported up to 83% of cases. Obtaining, securing and maintaining percutaneous access are critical to the success of a case. Consequently, a major concern when performing a PCNL is loss of access to the collecting system. This complication is increasingly seen in obese patients (possibly due to longer stone-to-skin distance), calyceal diverticular stones (narrow access), hypermobile kidneys, and supine positioning (longer stone-to-skin distance). Loss of access can occur when the access sheath is accidentally dislodged during introduction and removal of various instruments required during the operation.

### Sheaths

Aspects of the invention provide sheaths and methods of using the same. For example, an access sheath that can be introduced over both standard balloon and rigid dilators. Embodiments of the invention include an inflatable balloon at its tip that can be inflated with contrast upon entry of the access sheath into the renal pelvis. This would virtually eliminate the possibility of the access sheath becoming dislodged and prevent any chance of losing access to the kidney during the operation. In addition, it would help to seal the percutaneous access in place and prevent extravasation around the access sheath and potentially decrease intraoperative blood loss through a tamponade effect.

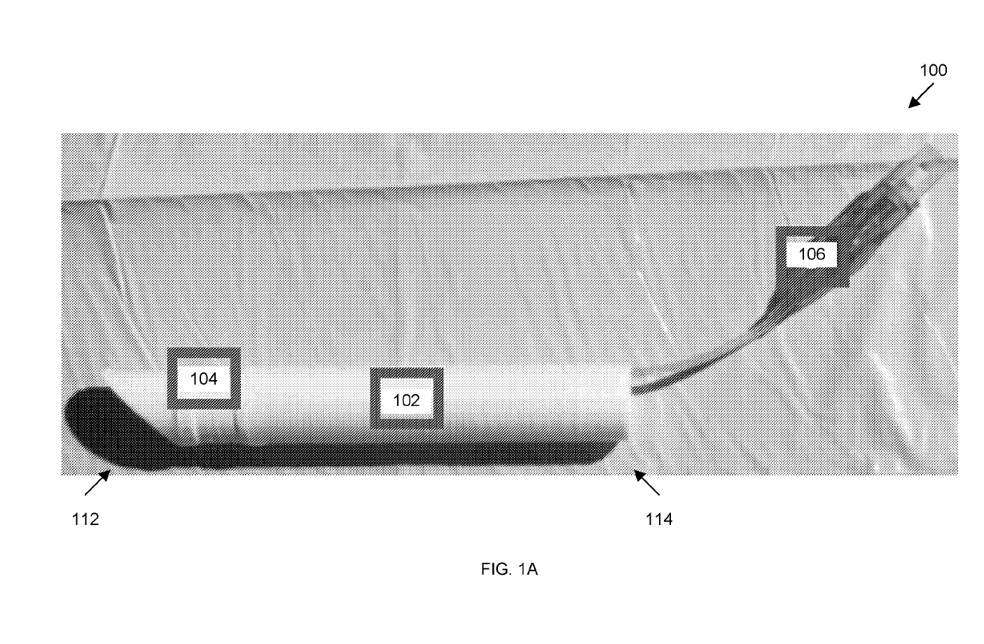

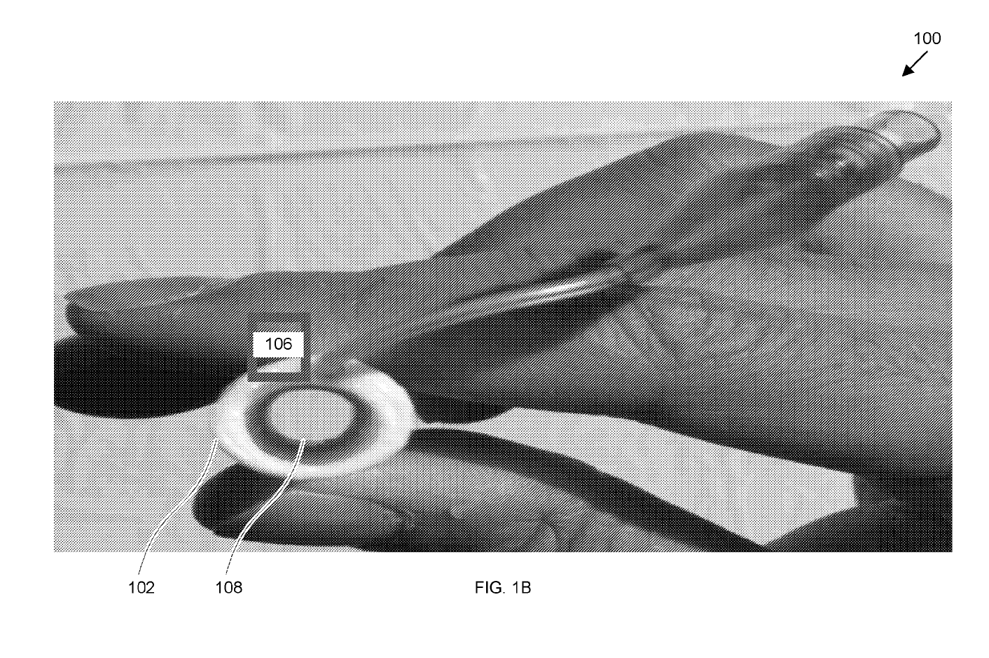

Referring now to FIGS. 1A and 1B, one embodiment of the invention provides a sheath 100 including a cylinder 102, a balloon 104, and an inflation port 106.

The cylinder 102 defines as central passage 108. Although a circular cross-section may be preferred for allowing accommodating various surgical instruments, other cross-sectional shapes can used.

Cylinder 102 can be fabricated from a variety of materials including metals (e.g., stainless steel), plastics, and the like using a variety of techniques including casting, molding, machining, thermomolding, thermosetting, injection molding, vacuum forming, additive manufacturing (also known as 3D printing), and the like.

Cylinder 102 preferably has sufficient rigidity to allow for advancement between a dilator and a tight puncture.

Cylinder can have a variety of internal and external dimensions. For example, the cylinder 102 can have an internal diameter or an external diameter selected from Table 1 below.

TABLE 1
Exemplary French Catheter Scale Values

| French Gauge | Diameter (mm) | Diameter (inches) |
|---|---|---|
| 3 | 1 | 0.039 |
| 4 | 1.333 | 0.053 |
| 5 | 1.667 | 0.066 |
| 6 | 2 | 0.079 |
| 7 | 2.333 | 0.092 |
| 8 | 2.667 | 0.105 |
| 9 | 3 | 0.118 |
| 10 | 3.333 | 0.131 |
| 11 | 3.667 | 0.144 |
| 12 | 4 | 0.158 |
| 13 | 4.333 | 0.170 |
| 14 | 4.667 | 0.184 |
| 15 | 5 | 0.197 |
| 16 | 5.333 | 0.210 |
| 17 | 5.667 | 0.223 |
| 18 | 6 | 0.236 |
| 19 | 6.333 | 0.249 |
| 20 | 6.667 | 0.263 |
| 22 | 7.333 | 0.288 |
| 24 | 8 | 0.315 |
| 26 | 8.667 | 0.341 |
| 28 | 9.333 | 0.367 |
| 30 | 10 | 0.393 |
| 32 | 10.667 | 0.419 |
| 34 | 11.333 | 0.445 |

In one embodiment, the cylinder 102 has an external diameter of up to about 36 French to accommodate a variety of use scenarios including mini-percutaneous procedures.

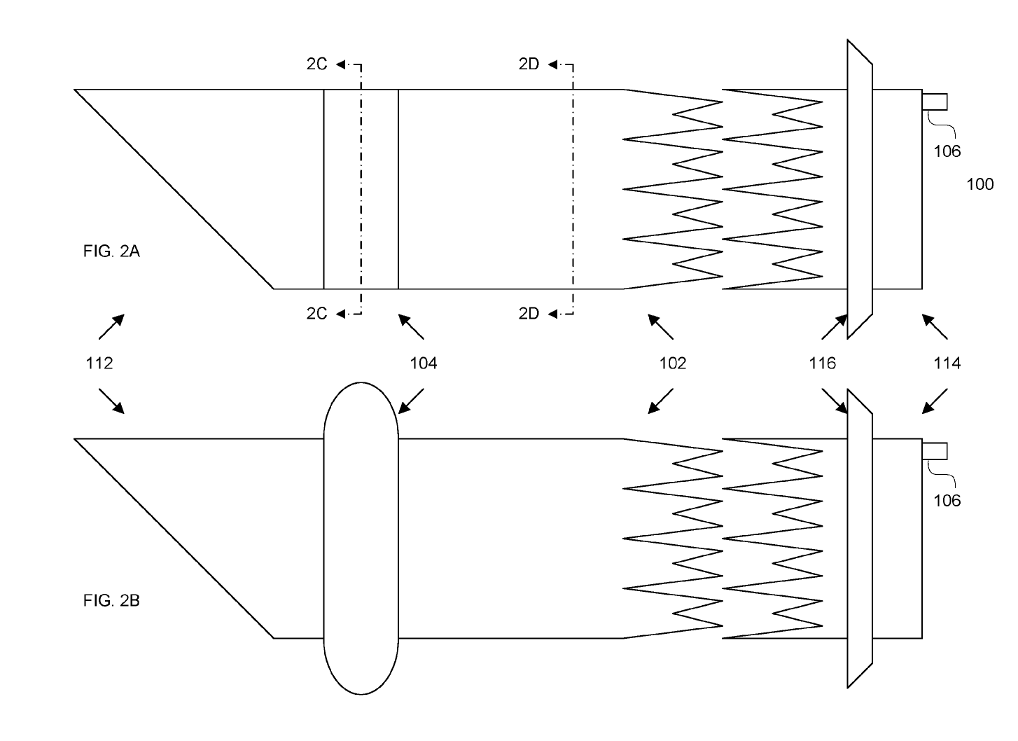

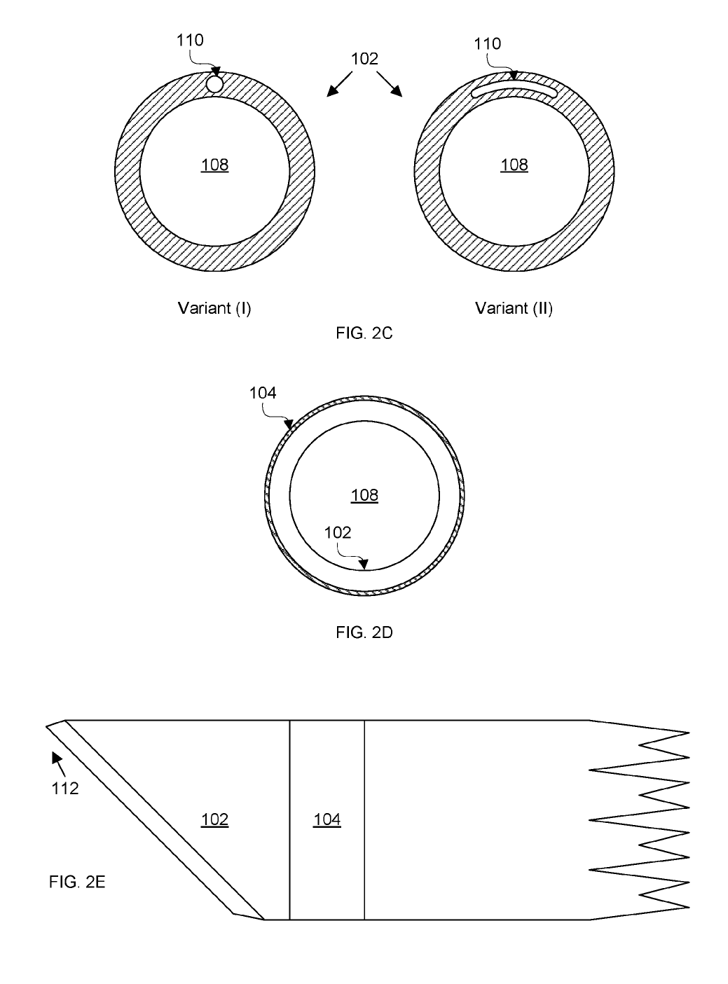

Although a beveled distal end 112 is depicted herein, distal end 112 can be square with respect to a central axis of the cylinder 102 or have other geometries. A variety of bevel angles can be employed such as 45° as depicted. The distal end 112 can be beveled with respect to a central axis as depicted in FIGS. 1A-2B and/or with respect to the external surface as depicted in FIG. 2E.

Balloon 104 can be constructed from a variety of elastomeric materials such as polyurethane, polyvinylchloride, latex, guayule latex, silicone rubber, and the like.

Balloon 104 can be coupled to inflation port 106 via one or more conduits. In one embodiment, cylinder 102 is fabricated to define a channel 110 as seen in FIG. 2C. Channel 110 can be a tube installed within cylinder 102 or can be fabricated in situ as cylinder 102 is formed (e.g., through additive manufacturing) or, for example, through boring or other machining after fabrication of cylinder 102.

The balloon 104 can have an uninflated cross-sectional diameter that is preferably the same or less than the cross-sectional diameter of the remainder of the cylinder 102 in order to minimize resistance and/or shearing during insertion of the sheath 100.

Inflation port 106 can be in fluid communication with an internal volume of balloon 104 (e.g., via channel 110) so that fluid introduced via inflation port 106 increases the pressure inside balloon 104, causing the balloon 104 to expand. Inflation port 106 can include or can be coupled to a valve such as a one-way valve such as those used in endotracheal tubes for anesthetic intubation. Using a 10-cc syringe, the one-way valve can be depressed and the balloon 104 on the distal end of the sheath 100 can be inflated.

Inflation port 106 can include or be coupled to a “pig tail” or other length of tubing and/or valves as depicted in FIGS. 1A and 1B.

Sheath 100 can also include a locking mechanism 116 slidable from distal end of sheath 100. Locking mechanism 116 can have an expanded diameter relative to sheath 100 in order to engage with the subject's skin around the puncture site. Locking mechanism 116 can include one or more elastomeric components to press against the skin. Locking mechanism 116 can include one or more screws and/or camming devices to retain the locking device 116 in place after advancement to contact the subject's skin.

### Methods of Performing Percutaneous Procedures

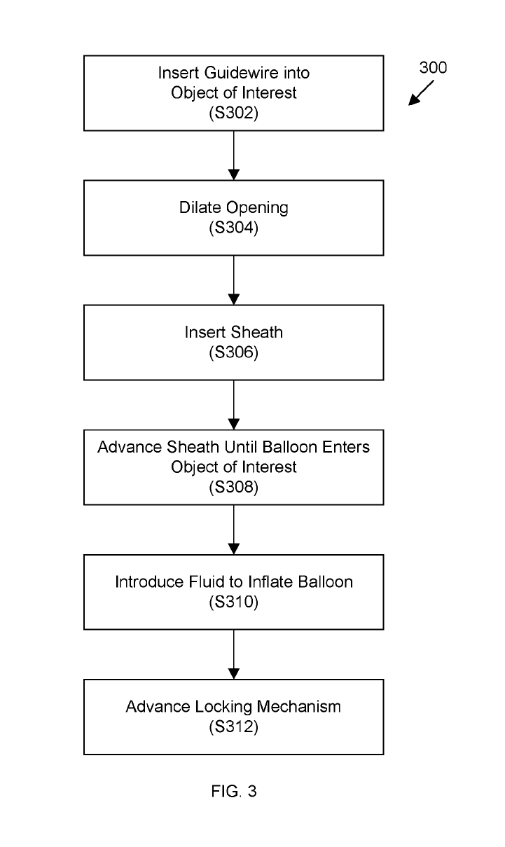

Referring now to FIG. 3, another aspect of the invention provides a method 300 of performing a percutaneous procedure. Although the method 300 may be described and depicted in the context of a percutaneous nephrolithotomy (PCNL) procedure, it is applicable to any other procedure in which visualization and procedures are completed percutaneously via a lumen, such as procedures within the bladder requiring percutaneous fragmentation (i.e., transvesical approach).

In step S302, a guidewire is inserted into an object of the interest such as the renal collecting system of a subject's kidney or the bladder. For example, the guidewire can be inserted through the subject's skin through a supracostal, intercostal, and/or infracostal puncture. Suitable guidewires are available under the BIWIRE® trademark from Cook Group, Inc. of Bloomington, Ind., under the SENSOR™ trademark from Boston Scientific Corporation of Natick, Mass., and under the ULTRATRACK® trademark from Olympus Medical Systems Corp. of Tokyo, Japan.

In step S304, an opening defined by a guidewire is dilated. The opening can be dilated by advancing a series of dilators having increasing diameters over the guidewire. In another embodiment, a balloon dilator is advanced over the guidewire and then inflated. Suitable balloon dilation catheters are available under the NEPHROMAX™ mark from Boston Scientific Corporation.

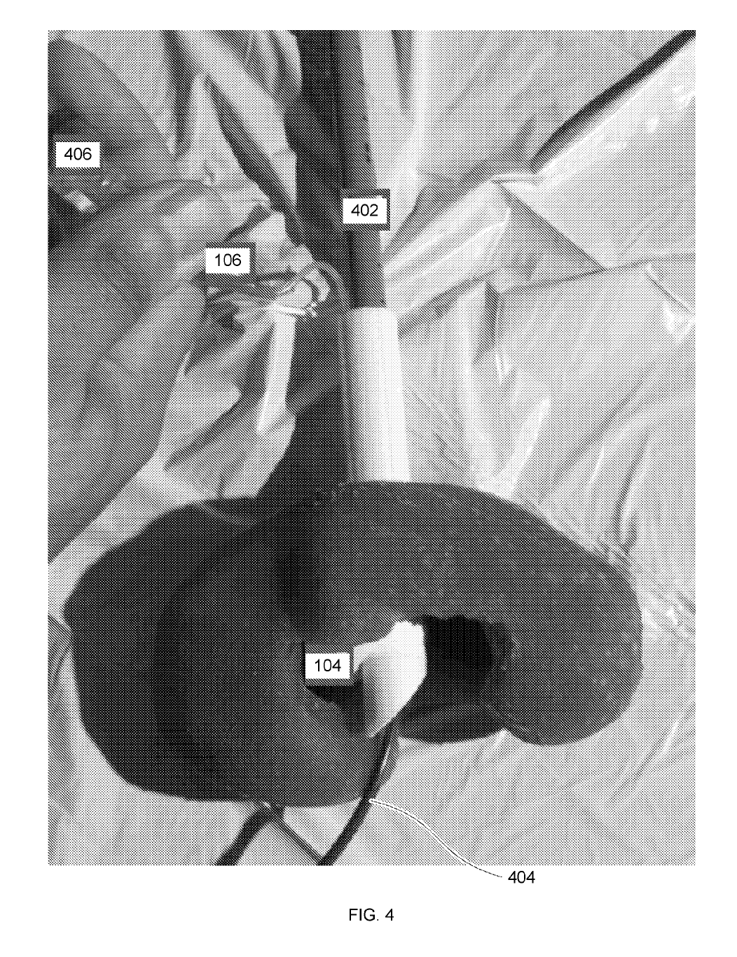

In step S306, a sheath is inserted into the opening. The sheath can be a sheath as described and depicted herein. For example, the sheath can include a balloon located at a distal end. The sheath can be advanced over a previously inserted dilator 402 and/or guidewire 404 through central passage 108 as depicted in FIG. 4.

In step S308, the sheath is advanced until the balloon enters the object of the interest. Sufficient depth can be verified, for example, through various imaging techniques.

In step S310, fluid is introduced into an inflation port to inflate the balloon. The fluid can be a gas (e.g., air, nitrogen, carbon dioxide, and the like) or a liquid (e.g., saline) and can be introduced using a syringe 406 as depicted in FIG. 4 or other pressure source. Inflation of the balloon retains the sheath within the object of interest as depicted in FIG. 4.

In step S312, a locking mechanism is advanced to secure the sheath relative to the subject's skin. In some embodiments, the locking mechanism is advanced to contact the skin surface and locked, which will minimize play in the sheath as the remainder of the procedure is performed. In other embodiments, force applies tension to the sheath (and therefore the inflated balloon and the object of interest) before locking in order to pull the object of interest close toward the skin surface.

### EQUIVALENTS

Although preferred embodiments of the invention have been described using specific terms, such description is for illustrative purposes only, and it is to be understood that changes and variations may be made without departing from the spirit or scope of the following claims.

In particular, sheath diameters and/or lengths are can be designed to address a variety of populations and skin-to-stone distances.

### INCORPORATION BY REFERENCE

The entire contents of all patents, published patent applications, and other references cited herein are hereby expressly incorporated herein in their entireties by reference.

1. A sheath comprising:
a cylinder defining a central passage;
a balloon located along an external surface of a distal end of the cylinder, the balloon lying substantially flush with or recessed from the external surface when deflated, but capable of projecting beyond the external surface when inflated; and
an inflation port located at a proximal end of the cylinder, the inflation port in fluid communication with the balloon.

2. The sheath of claim 1, further comprising:
a locking member slidably positioned along the external surface of the cylinder and adapted and configured to press against a subject's skin.

3. The sheath of claim 1, further comprising:
a one-way valve in communication with the inflation port, the one-way valve adapted and configured to retain a fluid and maintain the balloon in an inflated state.

4. The sheath of claim 1, wherein the distal end is beveled with respect to a central axis of the cylinder.

5. The sheath of claim 1, wherein the distal end is beveled with respect to the external surface of the cylinder.

6. The sheath of claim 1, wherein the cylinder has a cylindrical cross-section.

7. The sheath of claim 1, wherein the central passage has a diameter of about 26 French or greater.

8. The sheath of claim 1, wherein the central passage has a diameter of about 34 French or greater.

9. The sheath of claim 1, wherein the balloon is fabricated from a material selected from the group consisting of: polyurethane, polyvinylchloride, latex, guayule latex, and silicone rubber.

10. A method of performing a percutaneous procedure, the method comprising:
inserting a guidewire into an object of interest;
dilating an opening defined by the guidewire;
inserting the sheath of claim 1 into the opening;
advancing the sheath so that the balloon enters the object of interest; and
introducing a fluid into the inflation port to inflate the balloon, thereby retaining the sheath within the distal end of the sheath within the object of interest.

11. A method of performing a percutaneous nephrolithotomy (PCNL) procedure, the method comprising:
inserting a guidewire into a renal collecting system of a subject's kidney;
dilating an opening defined by the guidewire;
inserting the sheath of claim 1 into the opening;
advancing the sheath so that the balloon enters the renal collecting system; and
introducing a fluid into the inflation port to inflate the balloon, thereby retaining the sheath within the distal end of the sheath within the renal collecting system.

12. A kit comprising:
the sheath of claim 1; and
instructions for use.

13. A method of performing a percutaneous nephrolithotomy (PCNL) procedure, the method comprising:
inserting a guidewire through a subject's skin into a renal collecting system of the subject's kidney;
dilating an opening defined by the guidewire;
inserting a sheath into the opening, the sheath comprising:
a cylinder defining a central passage;
a balloon located along an external surface of a distal end of the cylinder, the balloon lying substantially flush with or recessed from the external surface when deflated, but capable of projecting beyond the external surface when inflated;
an inflation port located at a proximal end of the cylinder, the inflation port in fluid communication with the balloon; and
a locking member slidably positioned along the external surface of the cylinder and adapted and configured to press against a subject's skin;
advancing the sheath so that the balloon enters the renal collecting system;
introducing a fluid into the inflation port to inflate the balloon, thereby retaining the sheath within the distal end of the sheath within the renal collecting system; and
applying tension to the sheath to pull, via the balloon, the subject's kidney toward the subject's skin;
sliding the locking the locking member against the subject's skin; and
securing the locking member relative to the sheath.
